(19) United States
(12) Patent Application Publication
(10) Pub. No.: US 2021/0299058 A1
(43) Pub. Date: Sep. 30, 2021

(54) Intracellular Delivery System for mRNA Nucleic Acid Drugs, Preparation Method and Application Thereof

(71) Applicant: Shenzhen Neocura Biotechnology Corporation, Shenzhen (CN)

(72) Inventors: Youdong PAN, Shenzhen (CN); Gang LIU, Shenzhen (CN); Yi WANG, Shenzhen (CN); An XIAO, Shenzhen (CN); Ji WAN, Shenzhen (CN); Ying WEN, Shenzhen (CN); Qi SONG, Shenzhen (CN)

(73) Assignee: Shenzhen Neocura Biotechnology Corporation, Shenzhen (CN)

(21) Appl. No.: 16/903,523

(22) Filed: Jun. 17, 2020

(30) Foreign Application Priority Data
Mar. 26, 2020 (CN) 202010225030.8

(51) Int. Cl. A61K 9/51

(52) U.S. Cl. CPC A61K 9/5123; B82Y 5/00; A61K 9/5192

(57) ABSTRACT

A delivery system for mRNA nucleic acid drugs, a preparation method and an application thereof are provided. The delivery system includes lipid nanoparticles for loading one or more kinds of mRNA molecules, wherein the lipid nanoparticles are prepared from raw materials including an ionizable cationic lipid, a phospholipid auxiliary lipid, cholesterol, and a polyethylene glycol-derivatized phospholipid. In the mRNA nucleic acid drug targeted intracellular delivery system based on the non-viral carrier of the present invention, the mRNA is concentrated and loaded by the electrostatic interaction between the ionizable cationic lipid and the mRNA. Phospholipid auxiliary lipid component-mediated pH sensitivity and late endosomal escape enable mRNA nucleic acid drugs to be efficiently delivered to target cells and then released into the cytoplasm of the target cells for exerting a pharmacodynamic effect.

### CROSS REFERENCE TO THE RELATED APPLICATIONS

This application is based upon and claims priority to Chinese Patent Application No. 202010225030.8, filed on Mar. 26, 2020, the entire contents of which are herein incorporated by reference.

### TECHNICAL FIELD

The present invention belongs to the field of biomedicine, and more specifically, relates to an intracellular delivery system for mRNA nucleic acid drugs based on non-viral carriers, a preparation method and an application thereof, which can be produced and applied on a large scale.

### BACKGROUND

Messenger ribonucleic acid (mRNA) is a single-stranded ribonucleic acid that is synthesized by a polymerization where phosphodiester bonds are formed by four kinds of ribonucleoside triphosphates (A, U, G, and C) under the catalysis of RNA polymerase with one of the double strands of DNA (deoxyribonucleic acid) as a template. mRNA can carry and transmit the genetic information stored by DNA in a cell nucleus, which plays a key role in the conversion from genetic information to functional proteins. In the cytoplasm, immature mRNA can be modified into mature mRNA by capping, tailing, intron splicing, and other processes. Mature mRNA can accurately guide the synthesis of proteins in the cytoplasm. Compared to DNA, mRNA has a much smaller molecular weight, is conducive to transfection, and may not induce carcinogenic risk of insertion mutation caused by integration into host DNA. Therefore, mRNA, as a preventive and therapeutic drug, has great advantages and potential in the prevention and treatment of a variety of diseases.

For mRNA nucleic acid drugs, target functional genes or functional subunits of target genes are introduced into patients in the form of mRNA by molecular biological methods and proteins with specific functions are expressed after being subjected to a targeted intracellular delivery, escaping from late endosomes, intracellular translation, and modification after translation, which is a preventive and therapeutic method used to prevent (functional proteins or subunits activate the host immune system to produce corresponding humoral or cellular immune responses) or to treat diseases (expressed proteins or subunits have the function to treat diseases or regulate the expression of other genes). Compared with other methods, the advantage is that it can directly activate the body to produce functional antibodies or cellular immune responses against specific pathogens, repair pathogenic genes or correct abnormal gene expression at the molecular level, thus realizing the purpose of preventing and treating a variety of diseases. mRNA nucleic acid drugs show effects that traditional drugs cannot achieve, for example, monoclonal antibody drugs can merely act on the cell surface, while mRNA nucleic acid drugs can act not only on extracellular proteins, but also on intracellular proteins, even in the cell nucleus, and have accurate targeting. Among over 7000 diseases faced by human beings, about ⅓ of the diseases are caused by the abnormal expression (gene deletion, decrease or overexpression) of functional genes, such as hemophilia, Duchenne muscular dystrophy (DMD), cysticfibrosis, and severe combined immunodeficiency (SCID), which are almost clinically incurable. However, mRNA nucleic acid drugs are especially advantageous to these single-gene diseases. In the era of the popularization of personalized medicine and precision medicine, theoretically, all diseases caused by gene differences or gene abnormal expression can be accurately and effectively treated with mRNA nucleic acid drugs.

mRNA nucleic acid drugs have great advantages and potential in regulating gene expression and in the prevention and treatment of malignant diseases. However, numerous difficulties still exist in mRNA nucleic acid drugs in terms of the research and development, preparation and later drug administration. First of all, mRNA is a single strand, which is unstable in vitro and physiological conditions, as a result, mRNA is not only easily degraded by RNase in air or blood, but also easily cleared by mononuclear macrophages in liver, spleen and other tissues or organs. Second, since mRNA is negatively charged, it is difficult for mRNA to enter the cell through the cell membrane. Third, it is difficult for mRNA to escape from endosomes and enter the cytoplasm to play its role. In addition, uridine (U) in mRNA is easy to cause immunogenicity, in some cases, the immunogenicity may increase the potential side effects of mRNA drugs. Finally, the common occurrence of off-target effects is another big challenge in the preparation and administration of mRNA nucleic acid drugs. Therefore, the development of intracellular delivery systems for mRNA nucleic acid drugs is the key to its large-scale clinical application.

In recent years, nanotechnology has developed rapidly, and its application in biomedicine has attracted much attention. Nanoparticulate delivery systems are drug delivery systems whose particle diameter is at the nanometer level (1-1000 nm), which can concentrate and load drugs mainly through embedding, adsorption, encapsulation, covalent bonding or other manners and then targetedly deliver the drugs to specific organs or cells. Lots of studies have shown that nanoparticulate delivery systems can effectively overcome numerous difficulties faced by mRNA nucleic acid drugs in clinical application, such as easy degradation, difficult access to target organs or target cells, low efficiency of escaping from late endosomes, and so on. Currently, various forms of nanoparticulate delivery systems have been successfully developed, including protamines, polyplexes, dendrimers, inorganic nanoparticles, lipoplexes, etc., to improve the clinical therapeutic efficacy and reduce side effects of protein or chemotherapeutic drugs (such as paclitaxel, amphotericin B, etc.). However, up to now, nanoparticulate delivery systems specifically for mRNA nucleic acid drugs have not been successfully developed and widely used.

The development of mRNA nucleic acid drugs provides an effective means for the prevention and treatment of infectious diseases, cancer, diabetes and other major diseases. However, due to the poor capability to penetrate the cell membrane, the mRNA nucleic acid drugs do not have the capability of targeted transportation and are extremely unstable in the physiological environment. Therefore, the bottleneck of the research and development and large-scale clinical application of mRNA nucleic acid drugs lies in the development and commercialization of in vivo targeted delivery systems. To solve this important problem, the present invention provides a large-scale industrial preparation technique for preparing nanoparticles based on ethanol injection. This technique successfully realizes the efficient concentration and loading of mRNA nucleic acid drugs, and the delivery system for mRNA nucleic acid drugs is proved to have an excellent drug delivery capability at the cell level. It is believed that the delivery system of lipid nanoparticles (LNPs) for mRNA nucleic acid drugs will play an effective and extensive role in the prevention and treatment of a variety of diseases (including infectious diseases, tumors, diabetes, cardiovascular diseases, single-gene genetic diseases, etc.).

### SUMMARY

The objective of the present invention is to provide a composition for an efficient loading and effective intracellular delivery of mRNA nucleic acid drugs, a preparation method and procedure thereof, so as to solve the practical problems existing in the current mRNA nucleic acid drugs, such as poor stability, low intracellular delivery efficiency, failure in large-scale clinical application, and so on.

In a first aspect of the present invention, a delivery system for mRNA nucleic acid drugs is provided, including lipid nanoparticles for loading one or more kinds of mRNA, wherein the lipid nanoparticles are prepared from raw materials including an ionizable cationic lipid, a phospholipid auxiliary lipid, cholesterol, and a polyethylene glycol-derivatized phospholipid (PEG-lipid).

The ionizable cationic lipid enables the lipid nanoparticles to concentrate negatively charged mRNA molecules through electrostatic interactions. The phospholipid auxiliary lipid makes the lipid nanoparticles sensitive to pH changes (helping to escape from late endosomes), and improves membrane stability and mRNA transfection efficiency. The cholesterol allows lipid nanoparticles to regulate membrane fluidity. The polyethylene glycol-derivatized phospholipid can increase the hydrophilicity of the surface of lipid nanoparticles, reduce the non-specific adsorption of lipid nanoparticles to proteins, and reduce the immunogenicity of lipid nanoparticles in vivo.

In some embodiments of the present invention, the ionizable cationic lipid contains a monovalent or multivalent cationic amino group, and is at least one selected from the group consisting of

N1-[2-((1 S)-1-[(3-aminopropyl)amino]-4-[di(3-amino-propyl)amino]butyl carboxamido)ethyl]-3,4-di[oleyloxy]-benzamide (MVL5), 1,2-dioleoyl-3-trim ethyl ammonium-propane (chloride salt) (DOTAP), 1,2-di-O-octadecenyl-3-trimethylammonium propane (chloride salt) (DOTMA),
3-(dimethylamino)propyl(12Z,15Z)-3-[(9Z,12Z)-octadeca-9,12-dien-1-yl]henicosa-12,15-dienoate (DMAP-BLP), 3B—[N—(N′,N′-dimethylaminoethane)-carbamoyl]cholesterol hydrochloride (DC-Cholesterol.HCl), 1,2-dioleoyl-sn-glycero-3-ethylphosphocholine (EDOPC), and
2-(2,2-di((9Z,12Z)-octadeca-9,12-dienyl)-1,3-dioxolan-4-yl)-N,N-dimethylethanamine (Dlin-KC2-DMA). Preferably, the ionizable cationic lipid is MVL5.

In some embodiments of the present invention, the phospholipid auxiliary lipid is at least one selected from the group consisting of

1,2-di-(9Z-octadecenoyl)-sn-glycero-3-phosphoethanolamine (DOPE), 1,2-dioleoyl-sn-glycero-3-phospho-L-serine (DOP S), 1,2-dimyristoyl-sn-glycero-3-P (DMPC), and 1,2-dioleoyl-sn-glycero-3-phosphocholine (DOPC). Preferably, the phospholipid auxiliary lipid is DOPE.

In some embodiments of the present invention, the polyethylene glycol-derivatized phospholipid is at least one selected from the group consisting of

1,2-distearoyl-sn-glycero-3-phosphoethanolamine-N-[methoxy(polyethylene glycol)-2000] (DSPE-PEG 2000), 1,2-dimyristoyl-rac-glycero-3-methoxypolyethylene glycol-2000 (PEG-DMG 2000), and
1,2-dimyristoyl-sn-glycero-3-phosphoethanolamine-N-[methoxy(polyethylene glycol)-2000] (ammonium salt) (C14-PEG2000). Preferably, the polyethylene glycol-derivatized phospholipid is DSPE-PEG 2000.

In some embodiments of the present invention, the mRNA molecules are selected from intact mRNA molecules expressing functional proteins, therapeutic monoclonal antibodies, B cell epitopes, T cell epitopes, or tumor neoantigen peptides.

In some embodiments of the present invention, a molar ratio of the ionizable cationic lipid, the phospholipid auxiliary lipid, the cholesterol, and the polyethylene glycol-derivatized phospholipid is (5-60):(5-35):(25-70):(0.2-15), and preferably, is one selected from the group consisting of 10:26:61.5:2.5, 35:16:46.5:2.5, 40:10:40:10, 50:10:38.5:1.5, 50:10:39.5:0.5, and 57.1:7.1:34.3:1.4, and more preferably, is 10:26:61.5:2.5.

In some embodiments of the present invention, an average particle size of the lipid nanoparticles is 50-100 nm, preferably 80-90 nm.

In some embodiments of the present invention, under neutral conditions (pH 7.4), the Zeta potential of mRNA/lipid nanoparticles ranges from +30 mV to +35 mV.

In a second aspect of the present invention, a method for preparing the delivery system for mRNA nucleic acid drugs described in the first aspect is provided, including the following steps:

S1, completely dissolving the components for preparing the lipid nanoparticles in a first organic solvent for mixing, and then removing the first organic solvent by rotary evaporation to obtain a thin lipid membrane, and removing the residual first organic solvent in vacuum;

S2, dissolving the dried thin lipid membrane in a second organic solvent to obtain a liquid A;

S3, mixing an mRNA solution with the liquid A to obtain an mRNA/lipid nanoparticle suspension solution; and

S4, optionally, purifying, concentrating, and preserving the mRNA/lipid nanoparticles.

Preferably, the first organic solvent is chloroform; Preferably, the second organic solvent is anhydrous ethanol; Preferably, in step S3, a mass ratio of the mRNA molecules to the ionizable cationic lipid is 1:(10-20).

In some embodiments of the present invention, in step S1, conditions for forming and drying the thin lipid membrane are as follows: performing slow rotary evaporation at 0.06 Mpa (gauge pressure) and 30-35° C., until a uniform thickness thin lipid membrane is formed at the bottom of the round bottom flask, and then vacuum drying at −0.1 Mpa (gauge pressure) and 25-30° C. for 4-6 h.

In some embodiments of the present invention, in step S3, the mRNA solution includes a buffer for diluting an mRNA storage solution, and the buffer is preferably at least one selected from the group consisting of a sodium citrate buffer having a concentration of 50 mM and a pH of 4.0, a sodium citrate buffer having a concentration of 10 mM and a pH of 3.0, a sodium citrate buffer having a concentration of 10 and a pH of 4.0, and a sodium acetate buffer having a concentration of 50 mM and a pH of 5.0.

In some embodiments of the present invention, in step S3, a flow rate ratio of the mRNA solution to the liquid A and a total flow velocity of a mixing pipeline are controlled by a microfluidic method.

In some embodiments of the present invention, in step S3, the flow rate ratio of the mRNA solution to the liquid A is 1:(1-5).

In some embodiments of the present invention, in step S3, the total flow rate of the mixing pipeline of the mRNA solution and the liquid A is 1 ml/min-12 ml/min.

In some embodiments of the present invention, in step S4, the mRNA/lipid nanoparticles are purified by dialysis or tangential flow filtration.

Preferably, an interception pore size of a dialysis membrane is 10 kd.

Preferably, conditions of the dialysis for the mRNA/lipid nanoparticles includes: dialyzing twice in phosphate buffered saline (PBS) having a pH of 7.4 and a volume greater than or equal to 200 times volume of the mRNA/lipid nanoparticles, the first dialysis is performed at room temperature (25° C.) for 2-4 h, and the second dialysis is performed at a low temperature of 4° C. for 12-18 h, and a total duration of the first dialysis and the second dialysis not less than 18 h.

In some embodiments of the present invention, in step S4, the mRNA/lipid nanoparticles are concentrated by centrifugal ultrafiltration.

Preferably, an interception pore size of an ultrafiltration tube is 3 kd.

Preferably, the mRNA/lipid nanoparticles are concentrated by centrifuging with a fixed-angle rotor having an angle of 30-50 degrees and a weight of 14000 g at room temperature (25° C.) for 25-35 min.

In some embodiments of the present invention, in step S4, all the mRNA/lipid nanoparticles are filtered by a 0.22 μm filter membrane and a 0.1 μm filter membrane, and then sub-packaged for preservation.

Preferably, the mRNA/lipid nanoparticles are filtered by the 0.22 μm filter membrane for 5 times and then filtered by the 0.1 μm filter membrane for 3 times.

Preferably, the mRNA/lipid nanoparticles are preserved at −80° C. after purification, concentration and sub-packaging.

In a third aspect of the present invention, an application of the delivery system for mRNA nucleic acid drugs described in the first aspect in the preparation of drug delivery systems is provided.

The advantages of the present invention are as follows.

In the mRNA nucleic acid drug targeted intracellular delivery system based on the non-viral carrier of the present invention, the mRNA is concentrated and loaded by the electrostatic interaction between the ionizable cationic lipid and the mRNA. Phospholipid auxiliary lipid component-mediated pH sensitivity and late endosomal escape enable mRNA nucleic acid drugs to be efficiently delivered to target cells and then released into the cytoplasm of the target cells for exerting a pharmacodynamic effect. The phospholipid auxiliary lipid increases the capability of the mRNA/lipid nanoparticles to escape from the late endosomes, and improves the stability and the mRNA transfection efficiency of the mRNA/lipid nanoparticles. The intracellular delivery system for mRNA nucleic acid drugs with high delivery efficiency developed and prepared by the present invention is conducive to large-scale clinical application of these drugs.

The mRNA/lipid nanoparticles constructed by the present invention have high and stable intracellular delivery efficiency for the mRNA drugs, and significantly improve the prevention and treatment effects of the mRNA nucleic acid drugs.

### BRIEF DESCRIPTION OF THE DRAWINGS

FIG. 1 is a schematic diagram showing the principle of an intracellular delivery system for mRNA nucleic acid drugs of the present invention;

FIG. 2 shows a potential change of the mRNA/lipid nanoparticles in some embodiments of the present invention at different pH values;

FIG. 3 shows a qualitative analysis of encapsulation efficiency of mRNA/lipid nanoparticles in some embodiments of the present invention; and

FIG. 4A shows intracellular transfection efficiency of mRNA nucleic acid drugs according to some embodiments of the present invention by fluorescence microscope.

FIG. 4B shows intracellular transfection efficiency of mRNA nucleic acid drugs according to some embodiments of the present invention by flow cytometry.

### DETAILED DESCRIPTION OF THE EMBODIMENTS

The present invention is illustrated with the following specific embodiments. Those skilled in the art can easily understand the other advantages and effects of the present invention from the contents disclosed in this specification. The present invention can further be implemented or applied through different specific embodiments, and the details in this specification can also be modified or changed without deviating from the spirit of the present invention based on different viewpoints and applications.

Before further describing the specific embodiments of the present invention, it should be understood that the protection scope of the present invention is not limited to the following specific embodiments; furthermore, the terms used in the embodiments of the present invention are intended to describe the specific embodiments, rather than limit the protection scope of the present invention.

When a numerical range is presented in specific embodiments, it should be understood that unless otherwise stated in the present invention, two endpoints of each numerical range and any one value between the two endpoints can be selected for the present invention. Unless otherwise defined, all technical and scientific terms used in the present invention have the same meaning as those skilled in the technical field generally understand. In addition to the specific methods, devices and materials used in the embodiments, according to existing techniques known by those skilled in the art and the disclosure of the present invention, the present invention can further be realized by using any method, device and material of the existing techniques similar to or equivalent to the methods, devices and materials described in the embodiments of the present invention.

The materials, reagents, etc., used in the following embodiments, unless otherwise specified, are commercially available.

### Definition

The terms “prevention” and the like mean to exempt or significantly reduce the incidence of a disease by vaccinating healthy and normal people before the disease occurs.

The terms “treatment” and the like mean to alleviate or slow at least one symptom associated with a condition, or to slow or reverse the development of the condition, such as slowing or reversing the development of liver cancer.

The terms “endosome” and the like refer to a kind of membrane-wrapped vesicle structure, which can be divided into an early endosome and a late endosome. The early endosome is usually located on the outside of the cytoplasm. The late endosome is usually located on the inside of the cytoplasm, close to the cell nucleus. The late endosome contains a variety of hydrolases in the acidic internal environment.

The terms “protonation” and the like refer to a process by which an atom, molecule, or ion acquires proton (H⁺). It can be understood simply as a combination of a lone pair electron and a proton, that is, to combine one proton. Generally, this substance has lone pair electrons, and each of the lone pair electrons can bind one proton through a coordination bond.

The terms “B cell epitope” and the like refer to a sequence fragment or spatial conformation that can be specifically recognized and bound by B cell receptors (BCRs) or antibodies in antigenic molecules such as proteins, sugars, lipids, etc.

The terms “T cell epitope” and the like refer to a short peptide sequence presented by a major histocompatibility complex (MHC) molecule to a T cell receptor (TCR) after a protein antigen is processed by an antigen presenting cell (APC), which is generally a linear epitope.

The terms “tumor neoantigen” and the like refer to an antigen peptide fragment that exists on the surface of tumor cells in the form of MHC-peptide complex, which is produced by somatic gene mutation of tumor cells and closely bound to a major histocompatibility complex (MHC) molecule, and can be specifically recognized by the T cell receptor (TCR), thus activating the immune response of T cells.

As used in the present disclosure, the “mRNA/lipid nanoparticles” includes pharmaceutically effective amounts of mRNA, and pharmaceutically acceptable mRNA drug delivery carriers which can be used on a large scale in clinical applications.

As used in the present disclosure, the “transfected cell” is the cell in which an mRNA molecule has been introduced and a corresponding protein can be translated and expressed by the mRNA molecule.

In the following embodiments, the mRNA nucleic acid drug molecules are obtained by in vitro transcription. The cholesterol for regulating membrane fluidity is selected from a pharmaceutical-grade cholesterol derived from wool with a purity of more than 98%. The microfluidic control is mainly realized by NanoAssemblr®Benchtop nanoparticle synthesis system and the software PRECISION Nanosystems thereof.

### EMBODIMENTS

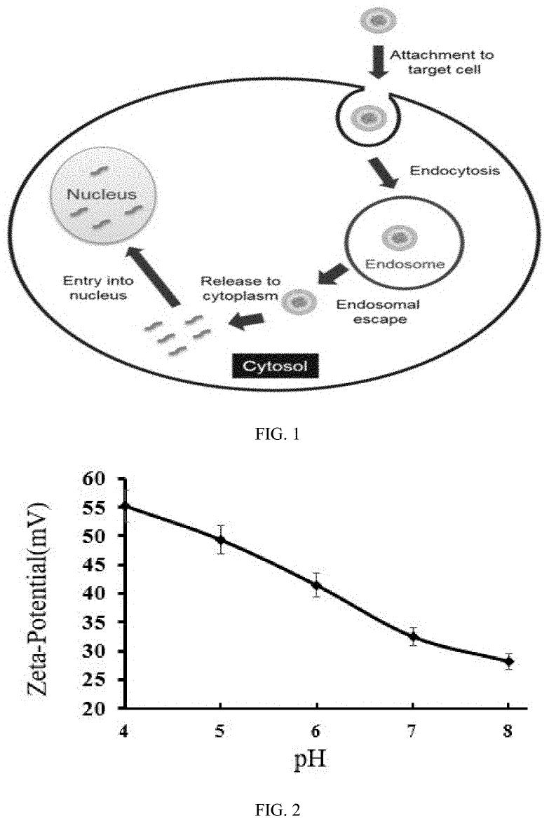

The principle of the intracellular delivery system for mRNA nucleic acid drugs prepared in the present invention is shown in FIG. 1. The delivery system for mRNA nucleic acid drugs is a complex formed by lipid nanoparticles encapsulating and loading the mRNA nucleic acid drugs. The lipid nanoparticles are composed of an ionizable cationic lipid, a phospholipid auxiliary lipid, cholesterol, and a polyethylene glycol-derivatized phospholipid in a certain ratio. Among them, the ionizable cationic lipid contains monovalent or multivalent cationic amino groups. These cationic amino groups can concentrate and load the mRNA molecules through electrostatic interaction with the negatively charged mRNA molecules. The phospholipid auxiliary lipid is sensitive to environmental pH changes, which help the lipid nanoparticles escape from late endosomes (The mRNA nucleic acid drugs are effectively delivered into the target cells and released in the acidic environment of the late endosomes having a specific pH in the target cells, so that the nucleic acid drugs are released into the cytoplasm for translation and expression into proteins, thereby exerting its function). Moreover, the phospholipid auxiliary lipid can also increase the membrane stability and mRNA transfection efficiency. The cholesterol can regulate the membrane fluidity. The polyethylene glycol-derivatized phospholipid (PEG-lipid) can increase the hydrophilicity of the surface of the lipid nanoparticles and reduce the non-specific adsorption of the lipid nanoparticles to proteins in serum or tissue fluid, thus reducing the immunogenicity of the lipid nanoparticles.

It is accepted that effective gene delivery requires a large molar charge ratio (cationic lipid/nucleic acid), but with the increase of the ionizable cationic lipid content, the damage to the cell membrane will increase, and the cytotoxicity of the prepared nanoparticles will increase as well. In this regard, novel MVL5 is selected as the ionizable cationic lipid in the present invention. One MVL5 molecule contains a multivalent cationic amino group, compared with ionizable cationic lipids containing monovalent cationic amino groups (such as EDOPC or DOTAP), in the process of preparing the lipid nanoparticles, less cationic lipids can achieve high mRNA cell transfection efficiency and significantly reduced cytotoxicity.

Successfully escaping from late endosomes is the key to drug delivery by the intracellular delivery system, which can prevent the drug molecules from being degraded by a large number of enzymes in the late endosomes. Related studies have shown that phosphatidylethanolamine combines with different kinds of unsaturated aliphatic hydrocarbons to form a phospholipid auxiliary lipid (such as DOPE). The phospholipid auxiliary lipid is negative in a neutral physiological environment (pH 7.4) with layered spatial structures. When the pH decreases (pH 5.0-6.0), phosphoethanolamine (PE) protonation makes the spatial conformation of the complex into hexagonal. The hexagonal complex is more destructive to the late endosome membrane. Taking advantage of this property, the phospholipid auxiliary lipid can help lipid nanoparticles escape from late endosomes under acidic conditions and prevent mRNA from being degraded by enzymes in the late endosomes.

DSPE-PEG2000 can increase the hydrophilicity of lipid nanoparticles, reduce the non-specific adsorption of lipid nanoparticles to proteins, and lower the probability of phagocytosis by mononuclear macrophages, because of its unique amphiphilic properties and spatial configuration.

In sum, in the present invention, components and their proportion in lipid nanoparticles are selected according to the sensitivity to pH changes, membrane stability, mRNA transfection efficiency, and loading effect of mRNA molecules, and the specific implementation paths for preparation, purification and concentration of the mRNA/lipid nanoparticles are optimized, aiming at developing an efficient targeted intracellular delivery system for mRNA nucleic acid drugs that can be used in large-scale clinical applications.

I. Main Reagents

| Main reagents in the present invention | Supplier |
|---|---|
| DOTMA | Avanti Polar |
| 1,2-di-O-octadecenyl-3-trimethylammonium propane | Lipids |
| (chloride salt) | |
| EDOPC | Avanti Polar |
| 1,2-dioleoyl-sn-glycero-3-ethylphosphocholine | Lipids |
| MVL5 | Avanti Polar |
| N1-[2-((1S)-1-[(3-aminopropyl)amino]-4-[di(3-amino- | Lipids |
| propyl)amino]butylcarboxamido)ethyl]-3,4-di[oleyloxy]- | |
| benzamide | |
| DOTAP | Avanti Polar |
| 1,2-dioleoyl-3-trimethylammonium-propane(chloride | Lipids |
| salt) | |
| DMAP-BLP | Avanti Polar |
| 3-(dimethylamino)propyl(12Z,15Z)-3-[(9Z,12Z)- | Lipids |
| octadeca-9,12-dien-1-yl]henicosa-12,15-dienoate | |
| DC-Cholesterol•HCl | Avanti Polar |
| (3β-[N-(N′,N′-dimethylaminoethane)- | Lipids |
| carbamoyl]cholesterol hydrochloride) | |
| Dlin-KC2-DMA | SuperLan |
| 2-(2,2-di((9Z,12Z)-octadeca-9,12-dienyl)-1,3-dioxolan-4- | |
| yl)-N,N-dimethylethanamine | |
| DOPS | Avanti Polar |
| (1,2-dioleoyl-sn-glycero-3-phospho-L-serine) | Lipids |
| DOPE | Avanti Polar |
| (1,2-di-(9Z-octadecenoyl)-sn-glycero-3- | Lipids |
| phosphoethanolamine) | |
| DMPC | Avanti Polar |
| (1,2-Dimyristoyl-sn-glycero-3-PC) | Lipids |
| DOPC | Avanti Polar |
| 1,2-dioleoyl-sn-glycero-3-phosphocholine | Lipids |
| DSPE-PEG 2000 | Avanti Polar |
| 1,2-distearoyl-sn-glycero-3-phosphoethanolamine-N- | Lipids |
| [methoxy(polyethylene glycol)-2000] | |
| PEG-DMG 2000 | Avanti Polar |
| 1,2-dimyristoyl-rac-glycero-3-methoxypolyethylene | Lipids |
| glycol-2000 | |
| C14-PEG2000 | Avanti Polar |
| 1,2-dimyristoyl-sn-glycero-3-phosphoethanolamine-N- | Lipids |
| [methoxy (polyethylene glycol)-2000] (ammonium salt) | |
| Cholesterol | Avanti Polar |
| | Lipids |
| Sodium citrate | Sigma-Aldrich |
| Sodium acetate | Sigma-Aldrich |
| RNase free deionized water | Thermo Fisher |
| | Scientific |
| Chloroform | Sigma-Aldrich |
| anhydrous ethanol | Sigma-Aldrich |
| 10xPBS (pH 7.4) | Sigma-Aldrich |
| 1xTris-borate-EDTA (TBE) buffer | BioRad |
| Gelred nucleic acid gel dye | Biotium |
| 6xloading Dye | Biotium |
| Agarose | BIOWEST |
| NaH₂PO₄•H₂O | Sigma-Aldrich |
| Na₂HPO₄•7H₂O | Sigma-Aldrich |
| Quant-iT ™ RiboGreen ™ RNA Assay Kit | Thermo Fisher |
| | Scientific |
| Triton-X100 | Sigma-Aldrich |

II. Main Instruments and Consumables

| Main instruments and consumables in the present | |
|---|---|
| invention | Supplier |
| NanoAssemblr ® Benchtop nanoparticle synthesis | Precision |
| system | Nanosystem |
| Rotary evaporator (R-1020) | Great Wall |
| Temperature control device (DL10-3000) | Great Wall |
| Vacuum acquisition and control device (SHB-B95) | Great Wall |
| Microplate reader (Infinite M200 Pro NanoQuant) | TECAN |
| Dynamic light scattering nanoparticle size analyzer | Malvern |
| (Zetasizer Pro) | Panalytical |
| Gel Imager (GelDoc XR+) | Bio-Rad |
| Flow cytometry (FACSCanto) | BD Biosciences |
| Inverted fluorescence microscope (CKX53) | Olympus |
| Vortex oscillator | SITIME |
| Magnetic stirrer | SITIME |
| Pipette | Eppendorf |
| −20° C. low temperature refrigerator | Zhongke Meiling |
| 4° C. low temperature refrigerator | Zhongke Meiling |
| −80° C. ultra-low temperature refrigerator | Thermo Fisher |
| | Scientific |
| Microwave oven | Zhongke Meiling |
| Nucleic acid electrophoresis system | BioRad |
| Filter tip | Axygen |
| Centrifuge tube | Axygen |
| Round bottom flask (25 ml) | Great Wall |
| Dialysis membrane | Thermo Fisher |
| | Scientific |
| Ultrafiltration centrifuge tube | EMD Millipore |
| Erlenmeyer flask (200 ml) | Great Wall |

III. Experimental Methods

1. Formation and Drying of Thin Lipid Membranes

Firstly, the four components (MVL5, DOPE, cholesterol, and DSPE-PEG 2000) were completely dissolved in chloroform, respectively, and then the dissolved solutions respectively containing 25 mg, 41.5 mg, 50.68 mg, and 14.06 mg of the four components (with a molar ratio of 10:26:61.5:2.5) were mixed uniformly, and then moved into a 25 mL round bottom flask for a slow rotary evaporation at 0.06 Mpa (gauge pressure) and 32° C. to remove chloroform until a layer of thin lipid membrane with uniform thickness is formed at the bottom of the round bottom flask. Subsequently, a vacuum drying is carried out at −0.1 Mpa (gauge pressure) and 28° C. for 5 h (to completely remove the residual chloroform).

2. Thin Lipid Membrane Dissolved in Anhydrous Ethanol

10 mL of anhydrous ethanol was added to the round bottom flask, and then the round bottom flask was moved to the magnetic stirrer and stirred evenly for 30 min until the thin lipid membrane disappeared (the thin lipid membrane was completely dissolved in the anhydrous ethanol).

3. Dilution of mRNA Solution

mRNA (100 μg/mL) dissolved in RNase-free deionized water was diluted with a sodium citrate buffer with a concentration of 50 mM and a pH of 4.0.

4. Preparation of mRNA/Lipid Nanoparticle Suspension Solution

The mixed components completely dissolved in anhydrous ethanol were quickly mixed with the diluted mRNA solution, which was controlled by Microfluidic mixers (NanoAssemblr) (operated by the software PRECISION Nanosystems thereof). The flow rate ratio (FRR) of ethanol phase to water phase was 1:3, and their flow rates were 5 mL/min and 15 mL/min, respectively. The total flow rate (TFR) in the mixing pipeline was 12 mL/min. The suspension solution of ethanol and water was obtained (the mass ratio of mRNA to ionizable cationic liposomes was 1:12.5 (w/w)).

5. Purification and Concentration of mRNA/Lipid Nanoparticles

The ethanol was removed by dialysis. The suspension solution obtained by the above step was dialyzed twice in PBS with a volume 200 times the volume of suspension solution and a pH of 7.4 (with 10 kd dialysis membrane). The first dialysis was performed at room temperature (25° C.) for 3 h, and the second dialysis was performed at 4° C. for 15 h. The mRNA/lipid nanoparticle suspension solution without ethanol was concentrated by centrifugal ultrafiltration to reach the final concentration of 1 μg/μl. The mRNA/lipid nanoparticle suspension solution was filtered by 0.22 μm membrane for 5 times and then filtered by 0.1 μm membrane for 3 times, and subsequently, was sub-packaged and preserved at −80° C.

IV. Experimental Results

1. Particle Size of Prepared mRNA/Lipid Nanoparticles

Different batches of samples filtered by 0.22 μm and 0.1 μm filter membranes were measured by the dynamic light scattering nanoparticle size analyzer. It was found that the average particle size of the mRNA/lipid nanoparticles was 85 nm. In a neutral environment (pH 7.4), the Zeta potential of the mRNA/lipid nanoparticles was +32.6 mV.

2. pH Sensitivity and Specificity Analysis of mRNA/Lipid Nanoparticles

The mRNA/lipid nanoparticles were mixed with phosphate buffer (PB) solutions of different pH and incubated at 37° C. for 30 min. The potential change of the mixture was measured by Zeta potentiometer. By measuring the surface potential changes of lipid nanoparticles at different pH, the stability of the mRNA/lipid nanoparticles in a neutral environment was reflected, and the ability of mRNA in the mRNA/lipid nanoparticles on escaping from late endosomes in the acidic environment was shown. The experimental results showed that the Zeta potential of the prepared mRNA/lipid nanoparticles was relatively stable in a neutral environment, but the Zeta potential of the surface of the mRNA/lipid nanoparticles increased sharply in an acidic environment. The results were shown in FIG. 2.

3. Qualitative Analysis of Encapsulation Efficiency of mRNA/Lipid Nanoparticles

(1) An appropriate amount of agarose was weighed and added into an appropriate amount of 1×TBE buffer to prepare 0.7% agarose nucleic acid gel.

(2) Appropriate amounts of the mRNA/lipid nanoparticle suspension solution and unencapsulated free mRNA were added into 6×loading Dye loading buffer, and then mixed and added into sample wells (with 250 ng of mRNA in each well). After adding the samples, the electrophoresis tank was covered and the power supply was turned on. The voltage of the power supply was controlled to maintain 60 V and the current was maintained above 40 mA. When the bromophenol blue band moved to about 2 cm from the front of the gel, the power supply was turned off and the electrophoresis was stopped.

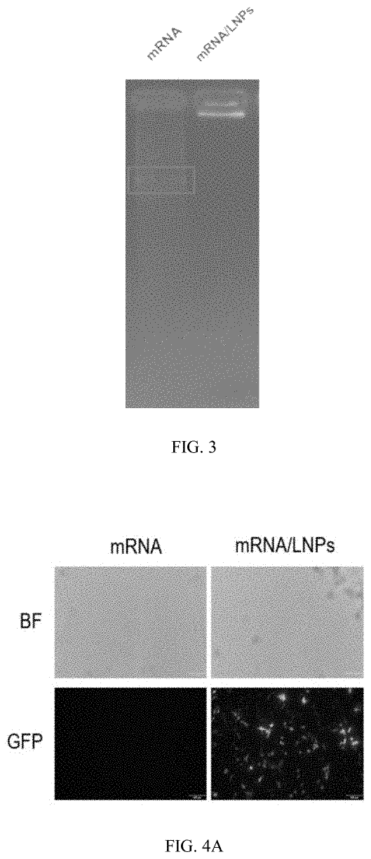

(3) After the electrophoresis, the nucleic acid gel was moved into a Gelred nucleic acid dye solution having a concentration of 0.5 μg/ml and stained in a dark environment at room temperature for 25 min. After staining, the gel was moved into the gel imager, and the stained mRNA was observed and photographed under ultraviolet light with the wavelength of 254 nm. The results showed that in the control group of unencapsulated free mRNA, there was mRNA staining in the electrophoresis lane (The free mRNA is shown in the box), while the mRNA encapsulated in the lipid nanoparticles (mRNA/LNPs) was completely blocked in the sample wells. (The results are shown in FIG. 3).

4. Accurate Quantitative Analysis of Encapsulation Efficiency of mRNA/Lipid Nanoparticles

(1) The prepared mRNA/lipid nanoparticle suspension solution and PBS (negative control, having the same volume of TE buffer) was diluted to 4 ng/μL with TE buffer in the kit to obtain an mRNA/lipid nanoparticle working solution.

(2) The mRNA/lipid nanoparticle working solution was further diluted with TE buffer (or TE buffer containing 2% of Triton-X100) to reduce its concentration to half, and mixed, and then kept at 37° C. for 10 min (TE buffer without Triton-X100 was used for the determination of unencapsulated free mRNA, while TE buffer containing 2% of Triton-X100 was used for the determination of the total mRNA in the mRNA/lipid nanoparticle working solution, where the total mRNA included the free mRNA and the mRNA encapsulated in the lipid nanoparticles). Each group of samples was set for three repetitions.

(3) After obtaining the standard curve of fluorescence intensity/concentration by calibrating with the standard sample, an appropriate amount of Quanti-iT™ RiboGreen RNA reagent nucleic acid dye was added to each group of samples for staining for 5 min according to the instructions of the kit. Each group of samples after dyeing was moved to the TECAN microplate reader for detection, and the software I-Control v.3.8.2.0 was used to accurately quantify the mRNA in the samples.

(4) The following formula was used to calculate the encapsulation efficiency of the mRNA in the lipid nanoparticles

Encapsulation efficiency=[1−m(free mRNA)/m(total mRNA)]×100%].

By measuring the concentrations of the free mRNA and total mRNA in three repeatedly diluted samples, the results showed that the encapsulation efficiency of the mRNA in the mRNA/lipid nanoparticles (mRNA/LNPs) prepared by this method was more than 98%.

5. mRNA Intracellular Transfection Efficiency of mRNA/Lipid Nanoparticle Drug Delivery System

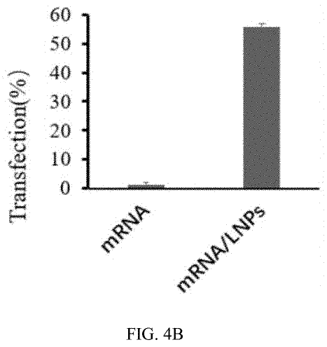

DC2.4 cells were inoculated into a 24-well plate (3×10⁵ cells/well). Free eGFP-mRNA (0.5 μg) and the mRNA/lipid nanoparticles (0.5 μg) were added to the cell culture medium, three wells for each group. After culturing for 48 h, the expression of the eGFP-mRNA in the DC2.4 cells was detected by the fluorescence microscope (the results are shown in FIG. 4A) and flow cytometry (the results are shown in FIG. 4B). These results showed that compared with unencapsulated free mRNA, the encapsulated mRNA can be effectively mediated into the cells and expressed at a high level by the mRNA/lipid nanoparticle (mRNA/LNPs) drug delivery system.

The preferred implementation ways and embodiments of the present invention are described in detail above, but the present invention is not limited to the above implementation ways and embodiments. Within the scope of knowledge possessed by those skilled in the art, various changes can also be made without departing from the conception of the present invention.

What is claimed is:

1. A delivery system for mRNA nucleic acid drugs, comprising lipid nanoparticles for loading one or more kinds of mRNA molecules, wherein the lipid nanoparticles are prepared from raw materials, and the raw materials comprise an ionizable cationic lipid, a phospholipid auxiliary lipid, cholesterol, and a polyethylene glycol-derivatized phospholipid.

2. The delivery system according to claim 1, wherein the ionizable cationic lipid contains a monovalent cationic amino group or a multivalent cationic amino group, and the ionizable cationic lipid is at least one selected from the group consisting of
N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-3-trimethyl ammonium-propane (chloride salt) (DOTAP), 1,2-di-O-octadecenyl-3-trimethylammonium propane (chloride salt) (DOTMA),
3-(dimethylamino)propyl(12Z,15Z)-3-[(9Z,12Z)-octadeca-9,12-dien-1-yl]henicosa-12,15-dienoate (DMAP-BLP), 3B—[N—(N′,N′-dimethylaminoethane)-carbamoyl]cholesterol hydrochloride (DC-Cholesterol.HCl), 1,2-dioleoyl-sn-glycero-3-ethylphosphocholine (EDOPC), and
2-(2,2-di((9Z,12Z)-octadeca-9,12-dienyl)-1,3-dioxolan-4-yl)-N,N-dimethylethanamine (Dlin-KC2-DMA),
and/or, the phospholipid auxiliary lipid is at least one selected from the group consisting of 1,2-di-(9Z-octadecenoyl)-sn-glycero-3-phosphoethanolamine (DOPE), 1,2-dioleoyl-sn-glycero-3-phospho-L-serine (DOP S), 1,2-dimyristoyl-sn-glycero-3-P (DMPC), and 1,2-dioleoyl-sn-glycero-3-phosphocholine (DOPC),
and/or, the polyethylene glycol-derivatized phospholipid is at least one selected from the group consisting of
1,2-distearoyl-sn-glycero-3-phosphoethanolamine-N-[methoxy(polyethylene glycol)-2000] (DSPE-PEG 2000), 1,2-dimyristoyl-rac-glycero-3-methoxypolyethylene glycol-2000 (PEG-DMG 2000), and
1,2-dimyristoyl-sn-glycero-3-phosphoethanolamine-N-[methoxy(polyethylene glycol)-2000] (ammonium salt) (C14-PEG2000),
and/or, the mRNA molecules are selected from intact mRNA molecules expressing functional proteins, therapeutic monoclonal antibodies, B cell epitopes, T cell epitopes, or tumor neoantigen peptides.

3. The delivery system according to claim 1, wherein a molar ratio of the ionizable cationic lipid, the phospholipid auxiliary lipid, the cholesterol, and the polyethylene glycol-derivatized phospholipid is (5-60):(5-35):(25-70):(0.2-15).

4. The delivery system according to claim 1, wherein an average particle size of the lipid nanoparticles is 50-100 nm; and/or,
under a neutral environmental condition, a Zeta potential of mRNA/lipid nanoparticles ranges from +30 mV to +35 mV.

5. A method for preparing the delivery system for mRNA nucleic acid drugs according to claim 1, comprising the following steps:
S1, completely dissolving the raw materials in a first organic solvent to obtain a mixture for mixing, and then removing the first organic solvent from the mixture by a rotary evaporation to obtain a thin lipid membrane, and removing a residual first organic solvent from the mixture by a vacuum drying to obtain a dried thin lipid membrane;
S2, dissolving the dried thin lipid membrane in a second organic solvent to obtain a liquid;
S3, mixing an mRNA solution with the liquid to obtain an mRNA/lipid nanoparticle suspension solution; and
S4, purifying and concentrating the mRNA/lipid nanoparticle suspension solution to obtain mRNA/lipid nanoparticles for preservation; wherein
the first organic solvent is chloroform;
the second organic solvent is anhydrous ethanol; and
in step S3, a mass ratio of mRNA molecules in the mRNA solution to the ionizable cationic lipid is 1:(10-20).

6. The method according to claim 5, wherein in step S1, the rotary evaporation is performed at a gauge pressure of 0.06 Mpa and 30-35° C., until a uniform thickness thin lipid membrane is formed at a bottom of a round bottom flask, and then the vacuum drying is performed at a gauge pressure of −0.1 Mpa and 25-30° C. for 4-6 h.

7. The method according to claim 5, wherein in step S3, the mRNA solution comprises a buffer for diluting an mRNA storage solution, and the buffer is at least one selected from the group consisting of a sodium citrate buffer having a concentration of 50 mM and a pH of 4.0, a sodium citrate buffer having a concentration of 10 mM and a pH of 3.0, a sodium citrate buffer having a concentration of 10 mM and a pH of 4.0, and a sodium acetate buffer having a concentration of 50 mM and a pH of 5.0.

8. The method according to claim 5, wherein in step S3, a flow rate ratio of the mRNA solution to the liquid and a total flow velocity of a mixing pipeline are controlled by a microfluidic method; and/or,
in step S3, the flow rate ratio of the mRNA solution to the liquid is 1:(1-5); and/or
in step S3, the total flow rate of the mixing pipeline of the mRNA solution and the liquid is 1 ml/min-12 ml/min.

9. The method according to claim 5, wherein in step S4, the mRNA/lipid nanoparticles are purified by a dialysis or a tangential flow filtration;
an interception pore size of a dialysis membrane for the dialysis is 10 kd;
a process of the dialysis for the mRNA/lipid nanoparticles comprises: dialyzing twice in a phosphate buffered saline (PBS) having a pH of 7.4 and a volume 200 times greater than or equal to a volume of the mRNA/lipid nanoparticles, a first dialysis is performed at room temperature (25° C.) for 2-4 h, and a second dialysis is performed at a low temperature of 4° C. for 12-18 h, with a total duration of the first dialysis and the second dialysis not less than 18 h; and/or
in step S4, the mRNA/lipid nanoparticles are concentrated by a centrifugal ultrafiltration;
an interception pore size of an ultrafiltration tube is 3 kd;
the mRNA/lipid nanoparticles are concentrated by centrifuging with a fixed-angle rotor having an angle of 30-50 degrees and a weight of 14000 g at room temperature of 25° C. for 25-35 min; and/or
in step S4, the mRNA/lipid nanoparticles are filtered by a 0.22 μm filter membrane for 5 times and a 0.1 μm filter membrane for 3 times, and then sub-packaged for preservation at −80° C.

10. A method of preparing a drug delivery system, comprising applying the delivery system according to any claim 1.

11. The delivery system according to claim 2, wherein a molar ratio of the ionizable cationic lipid, the phospholipid auxiliary lipid, the cholesterol, and the polyethylene glycol-derivatized phospholipid is (5-60):(5-35):(25-70):(0.2-15).

12. The delivery system according to claim 2, wherein an average particle size of the lipid nanoparticles is 50-100 nm; and/or,
under a neutral environmental condition, a Zeta potential of mRNA/lipid nanoparticles ranges from +30 mV to +35 mV.

13. The delivery system according to claim 3, wherein an average particle size of the lipid nanoparticles is 50-100 nm; and/or,
under a neutral environmental condition, a Zeta potential of mRNA/lipid nanoparticles ranges from +30 mV to +35 mV.

14. The method according to claim 5, wherein the ionizable cationic lipid contains a monovalent cationic amino group or a multivalent cationic amino group, and the ionizable cationic lipid is at least one selected from the group consisting of
N1-[2-((1S)-1-[(3-aminopropyl)amino]-4-[di(3-amino-propyl)amino]butyl carboxamido)ethyl]-3,4-di[oleyloxy]-benzamide (MVL5), 1,2-dioleoyl-3-trimethyl ammonium-propane (chloride salt) (DOTAP), 1,2-di-O-octadecenyl-3-trimethylammonium propane (chloride salt) (DOTMA),
3-(dimethylamino)propyl(12Z,15Z)-3-[(9Z,12Z)-octadeca-9,12-dien-1-yl]henicosa-12,15-dienoate (DMAP-BLP), 3B—[N—(N′,N′-dimethylaminoethane)-carbamoyl]cholesterol hydrochloride (DC-Cholesterol.HCl), 1,2-dioleoyl-sn-glycero-3-ethylphosphocholine (EDOPC), and
2-(2,2-di((9Z,12Z)-octadeca-9,12-dienyl)-1,3-dioxolan-4-yl)-N,N-dimethylethanamine (Dlin-KC2-DMA),
and/or, the phospholipid auxiliary lipid is at least one selected from the group consisting of 1,2-di-(9Z-octadecenoyl)-sn-glycero-3-phosphoethanolamine (DOPE), 1,2-dioleoyl-sn-glycero-3-phospho-L-serine (DOP S), 1,2-dimyristoyl-sn-glycero-3-P (DMPC), and 1,2-dioleoyl-sn-glycero-3-phosphocholine (DOPC),
and/or, the polyethylene glycol-derivatized phospholipid is at least one selected from the group consisting of
1,2-distearoyl-sn-glycero-3-phosphoethanolamine-N-[methoxy(polyethylene glycol)-2000] (DSPE-PEG 2000), 1,2-dimyristoyl-rac-glycero-3-methoxypolyethylene glycol-2000 (PEG-DMG 2000), and
1,2-dimyristoyl-sn-glycero-3-phosphoethanolamine-N-[methoxy(polyethylene glycol)-2000] (ammonium salt) (C14-PEG2000),
and/or, the mRNA molecules are selected from intact mRNA molecules expressing functional proteins, therapeutic monoclonal antibodies, B cell epitopes, T cell epitopes, or tumor neoantigen peptides.

15. The method according to claim 5, wherein a molar ratio of the ionizable cationic lipid, the phospholipid auxiliary lipid, the cholesterol, and the polyethylene glycol-derivatized phospholipid is (5-60):(5-35):(25-70):(0.2-15).

16. The method according to claim 5, wherein an average particle size of the lipid nanoparticles is 50-100 nm; and/or,
under a neutral environmental condition, a Zeta potential of mRNA/lipid nanoparticles ranges from +30 mV to +35 mV.

17. The method according to claim 6, wherein in step S3, the mRNA solution comprises a buffer for diluting an mRNA storage solution, and the buffer is at least one selected from the group consisting of a sodium citrate buffer having a concentration of 50 mM and a pH of 4.0, a sodium citrate buffer having a concentration of 10 mM and a pH of 3.0, a sodium citrate buffer having a concentration of 10 mM and a pH of 4.0, and a sodium acetate buffer having a concentration of 50 mM and a pH of 5.0.

18. The method according to claim 6, wherein in step S3, a flow rate ratio of the mRNA solution to the liquid and a total flow velocity of a mixing pipeline are controlled by a microfluidic method; and/or,
in step S3, the flow rate ratio of the mRNA solution to the liquid is 1:(1-5); and/or
in step S3, the total flow rate of the mixing pipeline of the mRNA solution and the liquid is 1 ml/min-12 ml/min.

19. The method according to claim 7, wherein in step S3, a flow rate ratio of the mRNA solution to the liquid and a total flow velocity of a mixing pipeline are controlled by a microfluidic method; and/or,
in step S3, the flow rate ratio of the mRNA solution to the liquid is 1:(1-5); and/or
in step S3, the total flow rate of the mixing pipeline of the mRNA solution and the liquid is 1 ml/min-12 ml/min.

20. The method according to claim 6, wherein in step S4, the mRNA/lipid nanoparticles are purified by a dialysis or a tangential flow filtration;
an interception pore size of a dialysis membrane for the dialysis is 10 kd;
a process of the dialysis for the mRNA/lipid nanoparticles comprises: dialyzing twice in a phosphate buffered saline (PBS) having a pH of 7.4 and a volume 200 times greater than or equal to a volume of the mRNA/lipid nanoparticles, a first dialysis is performed at room temperature (25° C.) for 2-4 h, and a second dialysis is performed at a low temperature of 4° C. for 12-18 h, with a total duration of the first dialysis and the second dialysis not less than 18 h; and/or
in step S4, the mRNA/lipid nanoparticles are concentrated by a centrifugal ultrafiltration;
an interception pore size of an ultrafiltration tube is 3 kd;
the mRNA/lipid nanoparticles are concentrated by centrifuging with a fixed-angle rotor having an angle of 30-50 degrees and a weight of 14000 g at room temperature of 25° C. for 25-35 min; and/or
in step S4, the mRNA/lipid nanoparticles are filtered by a 0.22 μm filter membrane for 5 times and a 0.1 μm filter membrane for 3 times, and then sub-packaged for preservation at −80° C.
